(19) United States
(12) Patent Application Publication
(10) Pub. No.: US 2024/0095909 A1
(43) Pub. Date: Mar. 21, 2024

(54) METHOD FOR AIDING IN THE DIAGNOSIS OF A CARDIOVASCULAR DISEASE OF A BLOOD VESSEL

(71) Applicant: NUREA, BORDEAUX (FR)

(72) Inventors: Florian BERNARD, BORDEAUX (FR); Romain LEGUAY, TALENCE (FR)

(21) Appl. No.: 18/257,050

(22) PCT Filed: Dec. 10, 2021
(86) PCT No.: PCT/EP2021/085268

§ 371 Date: Jun. 12, 2023

(30) Foreign Application Priority Data
Dec. 15, 2020 (FR) 2013257

(51) Int. Cl. G06T 7/00; A61B 5/02; G06T 7/11; G06T 7/136; G06T 7/62; G06V 10/764; G06V 10/77; G06V 10/82; G06V 20/64; G06V 20/70

(52) U.S. Cl. CPC G06T 7/0012; A61B 5/02014; G06T 7/11; G06T 7/136; G06T 7/62; G06V 10/764; G06V 10/7715; G06V 10/82; G06V 20/64; G06V 20/70; G06T 2200/04; G06T 2207/10028; G06T 2207/10081; G06T 2207/20072; G06T 2207/20081; G06T 2207/20084; G06T 2207/30101; G06T 2207/30172; G06V 2201/03

(57) ABSTRACT

A method for aiding in the diagnosis of a cardiovascular disease, comprising the following steps: providing a three-dimensional representation of a blood vessel of a patient; segmenting, by means of a classifier, the three-dimensional representation to obtain a segmented three-dimensional map; comparing the value of a plurality of voxels of the three-dimensional representation, the allocated labels on the three-dimensional map of the voxels being those of the blood vessel, with a predetermined threshold value, a label different from those of the blood vessel being allocated to each voxel with a value that exceeds the predetermined threshold value; determining the change in a geometric indicator of the blood vessel by means of the voxels of the three-dimensional representation, the allocated labels on the three-dimensional map of the aforementioned voxels being those of the blood vessel.

The invention relates to the field of the diagnosis of cardiovascular diseases or problems. More precisely, the invention relates to a method for aiding in the diagnosis of a cardiovascular disease of a blood vessel, and in particular of an abdominal aorta.

An abdominal aortic aneurysm (also called AAA) is a localized expansion, for example, welling or hypertrophy, of the wall of the aorta resulting in the formation of a pouch of variable size, also called thrombus, around the channel of the aorta wherein the blood circulates, also called lumen. This aneurysm can thus cause a restriction of the internal diameter of the lumen and/or an increase in the external diameter of the aorta and thus creates a risk of compression of the members close to the aorta, a risk of embolism or a risk of rupture of the aorta, which would lead to an internal hemorrhage.

It is known to detect and monitor the evolution of an aneurysm using medical imaging methods, such as Computed Tomography Angiography (CTA), or by Magnetic Resonance Imaging (MRI).

In the case of a CTA, a contrast agent or product is injected into the patient to improve the visibility of the aorta in the angiographies. Thus at the end of the CTA, the practitioner obtains a plurality of angiographies each showing a section of the aorta. In order to detect an aneurysm, the practitioner must examine all the angiographies to detect a large local variation of the diameter of the aorta. And he must then monitor the evolution of this diameter over time. This method has several drawbacks, and in particular those of being a manual, tedious and lengthy method and of being a practitioner-dependent method. Indeed, the step of calculating the diameter requires a selection of a particular image and a manual identification on this image to determine the diameter of the lumen, such that it depends on the practitioner's knowledge and is not easily reproducible from one consultation to another.

However, the evolution of the diameter of the aneurysm is one of the essential parameters in diagnosing and treating the aneurysm. In fact, the repair of an aneurysm is carried out through an angioplasty surgical operation, wherein the aneurysm is opened to implant a prosthesis, also called a stent, in the lumen of the aorta to expand it, or by an endovascular procedure, wherein a stent is deployed inside a blood vessel from a femoral artery. Since these operations are risky, the decision whether to proceed with such an operation is the result of a compromise between the risk of rupture and the risk of problems during the operation.

It has been found that the rupture rate of an aneurysm increases with the diameter of the aneurysm. In other words, the rupture risk is estimated as a function of the diameter of the lumen of the aorta. It should be noted that other geometric indicators of the aorta, such as its volume, allow this decision-making. It is thus necessary to be able to estimate these geometric indicators simply, reliably, quickly and reproducibly and in a manner that is not practitioner-dependent, in particular so that the measurements of these indicators, by two different practitioners or by the same practitioner during two different consultations, are consistent and allow reliable decision-making, which is not possible with the existing methods.

Moreover, it should be noted that the monitoring of the evolution of the aneurysm does not stop after the repair of the aneurysm. It is necessary to verify that, despite the placement of a stent, the diameter of the lumen is sufficient to allow blood circulation without generating new risks. Indeed, the cross section of the lumen can ultimately be reduced due to the stent, the circulation of the blood generating significant stresses on the walls of the aorta in this case. The same problem may arise when the aorta calcifies, for example in the case of aortic stenosis. Calcareous deposits appear in the lumen of the aorta, against the inner walls, which generates a narrowing of the circulating cross section of the aorta.

It is thus necessary, when estimating the diameter of the aorta, or

another geometric indicator of the aorta, to be able to detect elements capable of modifying the flow rate of the blood in the aorta and which would thus impinge on this indicator.

It should be noted that equivalent drawbacks are observed for other types of stenoses in other types of blood vessel.

The present invention lies in this context and aims to meet this need.

For these purposes, the invention relates to a method for aiding in the diagnosis of a cardiovascular disease of a blood vessel, comprising the following steps:
a. Providing a three-dimensional representation of a blood vessel of a patient, obtained by a medical imaging device;
b. Segmenting, by means of a classifier, said three-dimensional representation to obtain a segmented three-dimensional map of said three-dimensional representation, the classifier being arranged to estimate whether each voxel of the three-dimensional representation belongs to said blood vessel and to label this voxel as a function of this estimate, said segmented three-dimensional map being formed by the set of labels assigned by the classifier to the voxels of the three-dimensional representation;
c. Comparing the value of each voxel of a plurality of voxels of the three-dimensional representation, the allocated labels on the three-dimensional map of the voxels being those of the blood vessel, with a predetermined threshold value, a label different from those of the blood vessel being allocated to each voxel with a value that exceeds said predetermined threshold value;
d. Determining the change in a geometric indicator of the blood vessel along this blood vessel by means of the voxels of the three-dimensional representation, the allocated labels on the three-dimensional map of the aforementioned voxels being those of the blood vessel.

It will thus be understood that, owing to the invention, an automatic segmentation is carried out, by means of the classifier, of the three-dimensional representation of the blood vessel, so as to be able to exclusively select the voxels of this representation which actually correspond to the blood vessel, and in particular to the lumen of the blood vessel. The selection of these voxels then allows processing of the three-dimensional representation to be carried out in order to identify, by thresholding, the voxels classified in error by the classifier as belonging to the vessel whereas they correspond to a stent arranged in the vessel or belonging to calcification of the vessel. It is then possible to determine, simply, quickly, reliably and reproducibly, the actual diameter of the blood vessel or any other geographical indicator.

In one example embodiment of the invention, the three-dimensional representation provided comprises a stack of CT-metric angiographies of the patient's blood vessel. In order to obtain this stack, the patient is scanned, for example, helically, by an X-ray beam, so as to obtain a plurality of cross sectional images of the blood vessel according to different angular incidences of the irradiating beam. Each pixel of each cross sectional image therefore corresponds to a unit of volume of the patient, the thickness of which corresponds to the scanning resolution. It will thus be understood that the assembly of these images allows a digital reconstruction of a volume of points, in three dimensions, called voxels, forming a three-dimensional representation of the blood vessel. Each voxel is assigned a value proportional to the absorption of the X-rays by the corresponding scanned tissue or material. This value is measured in a Hounsfield units.

Other tomography methods may be employed within the scope of the invention to obtain several CT-metric angiographies, and in particular a conical beam volumetric imaging method, whereby a single rotary scan is carried out. It is also possible to envisage using other medical imaging techniques allowing a three-dimensional representation of the blood vessel to be obtained, such as magnetic resonance imaging.

Advantageously, at the end of the segmentation step, the three-dimensional map is formed by a set of voxels, each voxel of the three-dimensional map having the coordinates of one of the voxels of the three-dimensional representation and an intensity corresponding to the label assigned by the classifier to this voxel of the three-dimensional representation.

In one embodiment of the invention, the classifier is arranged to estimate, for each voxel of the three-dimensional representation, whether:
a. this voxel is outside the blood vessel, the classifier in this case allocating a first label to this voxel,
b. this voxel belongs to the lumen of the blood vessel, the classifier in this case allocating a second label to this voxel,
c. this voxel belongs to a tunica of the blood vessel, the classifier in this case allocating a third label to this voxel.

For example, the first label may be a zero value, the second label may be a value of 1, and the third label may be a value of 2. It will thus be understood that, in this example, the labels of the blood vessel are non-zero labels. This embodiment is particularly suitable for segmenting a three-dimensional representation obtained by means of computed tomography angiography wherein a contrast agent or product is injected into the patient to improve the visibility of the blood vessel in the angiographies. Indeed, the contrast product allows the classifier to distinguish the lumen from the blood vessel and the tissues forming the tunicas of the blood vessel.

It is also conceivable, in another embodiment of the invention, to carry out a segmentation of a three-dimensional representation obtained by means of computed tomography angiography without contrast product, the classifier in this case allocating only two labels, namely a first label for the voxels outside the blood vessel and a second label for the voxels of the blood vessel.

Advantageously, the segmentation step is implemented by a classifier implementing an automatic learning algorithm, in particular of the convolutional neural network type.

The three-dimensional representation of the blood vessel is formed by “scatter plots” each representing a well-defined part of the blood vessel. It is thus possible to define boundaries between these clouds, so that it is possible to allocate a label to the voxels of these portions. These boundaries are learned automatically, based on a set of reference three-dimensional representations, also called training set, the boundaries of each representation of this training set being known beforehand. The rules making it possible to decide whether or not to allocate a label to a voxel of a new three-dimensional representation are thus obtained from the training. Thus, a classifier implementing an automatic learning algorithm refers to a computer program whose role is to decide which label must be allocated to a voxel of a three-dimensional representation provided as input, according to the learned information. The label is determined by applying the decision rules (otherwise called knowledge base), which have themselves been previously learned on the training data.

Advantageously, the method comprises a prior step of supervised automatic training of the classifier, implemented by means of a plurality of predetermined three-dimensional representations. In other words, several predetermined three-dimensional representations forms a training set for the classifier, which thus automatically adjusts its decision rules (and therefore its boundaries), as a function of the label that it allocates to each voxel of each three-dimensional representation of the training set and the actual label of this voxel.

If desired, the method may comprise a prior step of increasing the training set wherein new three-dimensional representations are generated, from the three-dimensional representations of the training set, which are distinct from all the three-dimensional representations of the training set. For example, this generation of new three-dimensional representations may be carried out by modifying one of the three-dimensional representations of the training set so as to obtain at least one new three-dimensional representation that is distinct from all the three-dimensional representations of the training set. This modification can be carried out in particular by means of one or more of the following types of changes: degradation of all or part of the initial three-dimensional representation, change of resolution, addition of a noise, offset in one or more dimensions, rotation.

Advantageously, the classifier is a convolutional neural network, comprising a contraction path and an expansion path, wherein the contraction path comprises a plurality of convolution layers each associated with a correction layer arranged to implement an activation function and downsampling layers, each downsampling layer being followed by at least one convolution layer, wherein the expansion path comprises a plurality of convolution layers and upsampling layers, each upsampling layer being followed by a convolution layer. The downsampling layers are also called “pooling” layers. If necessary, the output of each upsampling layer can be concatenated, before entering the next convolution layer, to the feature map arising from a corresponding convolution layer of the contraction path through a connection hop between the contraction path and the expansion path. Such a convolutional neural network is for example known as “U-Net.”

In one embodiment of the invention, the segmentation step comprises the segmentation by means of the classifier of three axial, sagittal and coronal cross sections of said three-dimensional representation to obtain three segmented two-dimensional maps and a step of combining the two-dimensional maps to obtain said three-dimensional map.

According to this example, said three-dimensional representation can be scanned along three vertical, horizontal and transverse axes to obtain a plurality of images of axial, sagittal and coronal cross sections of the three-dimensional representation, each cross sectional image being segmented, by means of the classifier, so as to obtain a segmented two-dimensional map of said image, the classifier being arranged to estimate whether each pixel of the image belongs to said blood vessel and to label this pixel as a function of this estimate. Because of the scanning, each label associated with a pixel can be repositioned in space, so as to recombine all of the two-dimensional maps to form voxels of labels, this set of voxels of labels then forming the three-dimensional map. Advantageously, when two labels, associated with pixels of images of cross sections obtained along two distinct axes and which correspond to a same voxel, differ, the highest value label is assigned to this voxel.

In a non-limiting embodiment of the invention, the contraction path can receive as input an image of size 256×256 pixels and comprise a plurality of contraction blocks, in particular four, each comprising two convolution layers of standard type followed by a downsampling layer, the first convolution layer of the first contraction block receiving said image and the first convolution layer of the following contraction blocks receiving as input the feature map from the downsampling layer of the preceding block. If desired, each convolution layer may comprise a plurality of convolutional kernels of 3×3 dimensions and a stride of 1. For example, the number of convolutional kernels of each convolution layer of the first contraction block can be 64, and the number of convolutional kernels of each convolution layer of the following contraction blocks can be twice the number of convolutional kernels of each convolution layer of the preceding block. If desired, each correction layer associated with a convolution layer can be a rectified linear unit layer. If desired, each downsampling layer may comprise a mask for selecting a maximum value (max pooling) of dimensions 2×2 and a stride of 2.

Advantageously, the contraction path and the expansion path can be connected to each other by a plurality of convolution layers of successive standard type, in particular two, each comprising a plurality of convolutional kernels of dimensions 3×3 and a stride of 1, the number of convolutional kernels of each of these convolution layers being twice the number of convolutional kernels of each convolution layer of the last contraction block.

In this example, the expansion path can receive as input the feature map from the last convolution layer and comprise a plurality of expansion blocks, in particular four, each comprising an upsampling layer followed by two convolution layers of standard type, the upsampling layer of the first expansion block receiving said feature map and the upsampling layer of the following expansion blocks receiving as input the feature map from the last convolution layer of the preceding block. For example, each upsampling layer can be arranged to perform a transposed convolution operation which performs an upsampling and an interpolation from a plurality of convolutional kernels of dimensions 3×3 and with a stride of 2. Still advantageously, the number of convolutional kernels of the upsampling layer and of each convolution layer of the first expansion block can be identical to the number of convolutional kernels of each convolution layer of the last contraction block, and the number of convolutional kernels of the upsampling layer and of each convolution layer of the following expansion blocks may be half of the number of convolutional kernels of each convolution layer of the preceding block. If necessary, the first convolution layer of an expansion block can receive as input a concatenation of the feature map from the upsampling layer of this expansion block and of the feature map, optionally trimmed, coming from the last convolution layer of the contraction block having the same number of convolutional kernels.

Finally, according to this example, the classifier may comprise a last convolution layer, able to transform the feature maps from the expansion path into a label mask, by allocating the class having the highest probability to each pixel of the cross sectional image which is segmented. For example, this convolution layer may comprise a convolutional kernel of dimensions 1×1, associated with a normalized exponential-type correction layer (“Softmax”).

In this example, the classifier may for example be a convolutional neural network of the “U-Net 2D” type able to segment images, the hyperparameters of this classifier, and in particular the weights of the convolutional kernels of all the convolutional layers and upsampling layers are optimized during the prior training step, in particular by a gradient descent method.

In another embodiment of the invention, which the segmentation step can be implemented directly on the three-dimensional representation to obtain said three-dimensional map.

If necessary, each convolution layer may comprise a convolutional kernel of dimensions 3×3×3 or of dimensions 3×3, and a stride of 1. If desired, each correction layer may be a rectified linear unit layer. If desired, each sampling layer may comprise a mask for selecting a maximum value of dimensions 2×2×2 or of dimensions 2×2 and a stride of 2. If desired, each upsampling layer can be arranged to perform a transposed convolution operation which performs an upsampling and an interpolation from a convolutional kernel of dimensions 3×3×3 or of dimensions 2×2. In this example, the classifier may for example be a convolutional neural network of the “U-Net 3D” type able to segment a stack of images.

Advantageously, the method comprises, at the end of the segmentation step and prior to the comparison step, a step of confirming and correcting the labels allocated by the classifier to the voxels of the three-dimensional representation. This confirmation and correction step allows correction of the false positives and false negatives introduced by the classifier during the segmentation step, so as to further increase the reliability of the method according to the invention.

According to one embodiment of the invention, the confirmation and correction step comprises morphological operations carried out on the three-dimensional map and in particular operations of the erosion and expansion type. The erosion-type operations allow elimination of crenelated aspects that may have the contours of zones of the three-dimensional map whose voxels have a same label at the end of the segmentation, these crenelated aspects being inconsistent with the morphology of a blood vessel. The expansion-type operations allow grouping together of zones of the three-dimensional map which are neighbors but distant and whose voxels nevertheless have a same label at the end of the segmentation, the tunicas and the lumen of a blood vessel normally being continuous.

According to an alternative or cumulative example of the invention, the step of confirmation and correction may comprise a step of propagating the label of the voxels of a zone of the three-dimensional map to voxels of another zone whose label is different, the zones formed by the voxels of the three-dimensional representation corresponding to these zones of the three-dimensional map having a substantially identical texture. If necessary, this propagation step may comprise a step of determining averages intensity gradients and/or average intensities of voxels of the three-dimensional representation in order to determine zones of this three-dimensional representation whose textures are substantially identical. For example, two zones can be considered to have identical textures if the averages of the intensity gradients of these zones and/or if the averages of the intensities of these zones differ, in absolute value, by a value less than a threshold function of the standard deviation of these intensity gradients and/or of these intensities.

For example, it is possible to determine averages of intensity gradients and/or average intensities of a first zone of voxels of the three-dimensional representation to which the first label was allocated and a second zone of voxels of the three-dimensional representation to which the second label was allocated, the voxels of these first and second zones being located on either side of a boundary separating the first label zone from the second label zone in the three-dimensional map. In the case where the first zone and the second zone have an identical texture, the second label can be propagated to the voxels of the first zone.

It will also be possible, and in particular following the preceding propagation, to determine intensity gradients and/or average intensities of a second zone of voxels of the three-dimensional representation to which the second label has been allocated and a third zone of voxels of the three-dimensional representation to which the third label has been allocated, these second and third zones being located on either side of a boundary separating the second label zone from the third label zone in the three-dimensional map. In the case where the second zone and the third zone have an identical texture, the third label can be propagated to the voxels of the second zone.

In one embodiment of the invention, the comparison step comprises:
a. a first sub-step of comparing the value of each voxel of a plurality of voxels of the three-dimensional representation, the allocated labels on the three-dimensional map of the voxels being those of the blood vessel, with a first predetermined threshold value, a first label associated with a stent being allocated to each voxel with a value that exceeds said first predetermined threshold value;
b. a second sub-step of comparing the value of each voxel of a plurality of voxels of the three-dimensional representation, the allocated labels on the three-dimensional map of the voxels being those of the blood vessel, with a second predetermined threshold value that is less than the first threshold value, a second label associated with calcification being allocated to each voxel with a value that exceeds said second predetermined threshold value.

According to these features, it is thus possible to detect, in a first step, the voxels corresponding to a stent arranged in the blood vessel, then in a second step, the voxels corresponding to a calcification of the blood vessel, the latter having an intensity lower than that of the former. For example, the first threshold value may be 1500, and the second threshold value may be 500. If necessary, the comparison step may comprise a step of confirming and correcting the first and second labels, corresponding respectively to a stent and to a calcification, allocated to the voxels of the three-dimensional representation at the end of the comparison sub-steps, for example by means of morphological or label propagation operations as described above. Owing to these features, it is thus possible to determine the change of a geometric indicator reflecting the actual flow rate of the blood in the blood vessel, and not only the theoretical flow rate.

Advantageously, the comparison step is implemented for a plurality of voxels whose allocated labels on the three-dimensional map are those of the lumen of the blood vessel and are located at a boundary of the three-dimensional map between the labels of the lumen and the labels of the tunicas of the blood vessel. Thus, the comparison step is simplified, insofar as a stent is intended to come against the inner walls of a blood vessel defining its lumen so that calcification forms normally in the lumen and against these inner walls.

In one embodiment of the invention, the step of determining the change of a geometric indicator of the blood vessel is a step of determining the change of the diameter of the blood vessel. If necessary, this determining step comprises a step of estimating a graph passing through the entire blood vessel and each point of which is the barycenter of the voxels located in a cross section of the three-dimensional representation locally orthogonal to the graph and the labels of which are those of the blood vessel; a local diameter of the blood vessel is determined as a function of each of the points of the graph. “Graph” is understood to mean a succession of points where each point is connected to at least one other point of the graph, so that it is possible to travel the blood vessel from end to end by means of the graph.

For example, a first point of the graph may be estimated by determining the barycenter of the voxels located in the highest cross-section of the three-dimensional representation and the labels of which are those of the blood vessel. If necessary, each following point of the graph may be determined by estimating the barycenter of the intersection between the three-dimensional representation and a sphere, centered on the preceding point of the graph and with a radius greater than or equal to the smallest radius encompassing the voxels located in the cross section of the three-dimensional representation passing through this preceding point of the graph and whose labels are those of the blood vessel, until the entire three-dimensional representation has been traveled. For example, the radius of the sphere may be equal to said smallest radius plus two voxels. This algorithm has the advantage of being particularly robust with respect to the branches that a blood vessel may have. Indeed, in the case of a branching of the blood vessel, the algorithm will identify two intersections between the sphere and each branch of the blood vessel, so as to then be able to travel each of these branches.

If desired, said smallest radius may be the one encompassing the voxels located in the cross section of the three-dimensional representation passing through the preceding point of the graph and the labels of which are those of the lumen of the blood vessel. As a variant, said smallest radius may be the one encompassing the voxels located in the cross section of the three-dimensional representation passing through the preceding point of the graph and the labels of which are those of the lumen of the blood vessel or of a stent or a calcification.

Advantageously, the determining step further comprises a step of estimating a point cloud, each point of the point cloud being the point locally furthest from a boundary of the voxels of the three-dimensional representation of the blood vessel and the labels of which are those of the blood vessel, and a step of correcting the points of the graph using the point cloud. For example, each point may be determined by estimating the discontinuities of the gradient of the signed distance function to the walls of the blood vessel, in particular by estimating the barycenter of the discontinuity points of this gradient, these walls being able to be actualized by determining a boundary of demarcation of the three-dimensional map between the first and second labels or by determining a boundary of demarcation of the three-dimensional map between the second and third labels. If necessary, the step of correcting the points of the graph can be carried out by minimizing the distance between the points of the point cloud and the points of the graph.

For example, for each point of the graph, it is possible to select the point of the point cloud located at the smallest distance from this point of the graph, for example by means of a least squares method, said point of the graph being replaced by said point of the selected point cloud. If desired, said replacement may be conditioned by a stiffness constraint of the graph. For example, each branch of the graph can be represented by a regular polynomial, the replacement of a point of this branch by a point of the selected point cloud being conditioned on the fact that this selected point can be substantially represented by this polynomial.

The invention also relates to a computer program comprising program code which is designed to implement the method according to the invention.

The invention also relates to a data medium on which the computer program according to the invention is recorded.

The present invention is now described with the aid of examples that are purely illustrative and in no way limiting on the scope of the invention, and based on the attached drawings, wherein drawings the various figures shown:

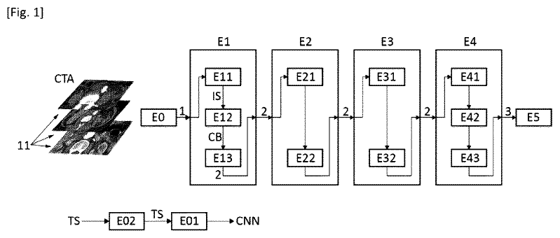

FIG. 1 shows, schematically and partially, a method for aiding in the diagnosis of a cardiovascular disease of a blood vessel according to one embodiment of the invention;

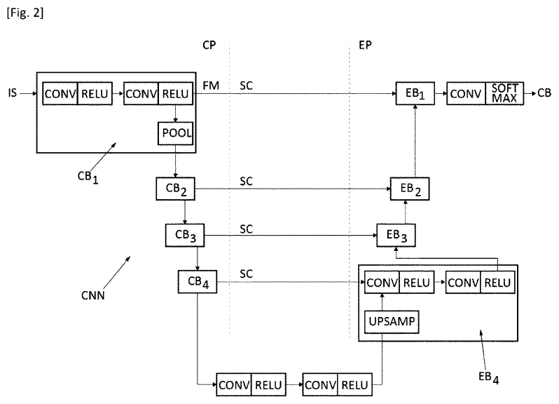

FIG. 2 shows, schematically and partially, a convolutional neural network used in the method of FIG. 1;

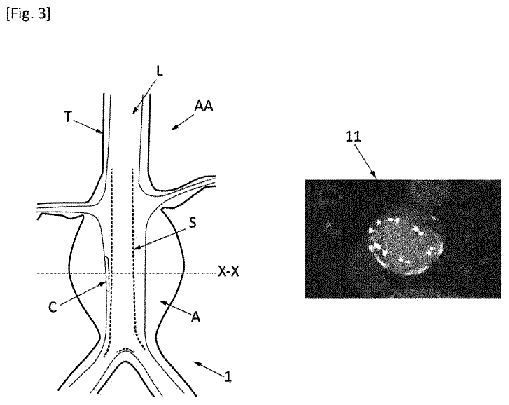

FIG. 3 shows, schematically and partially, a step of the method of FIG. 1;

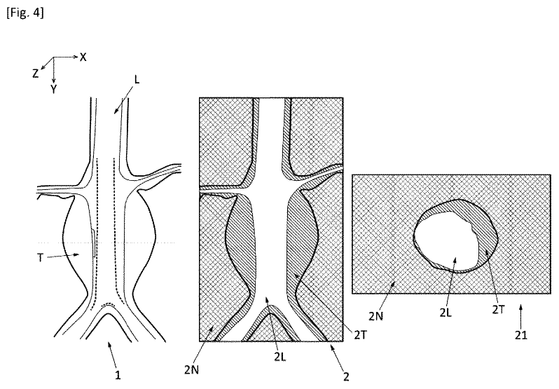

FIG. 4 shows, schematically and partially, another step of the method of FIG. 1;

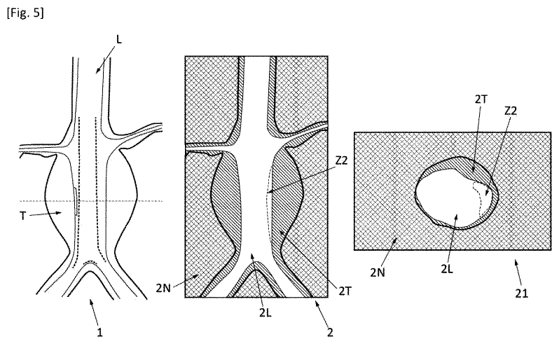

FIG. 5 shows, schematically and partially, another step of the method of FIG. 1;

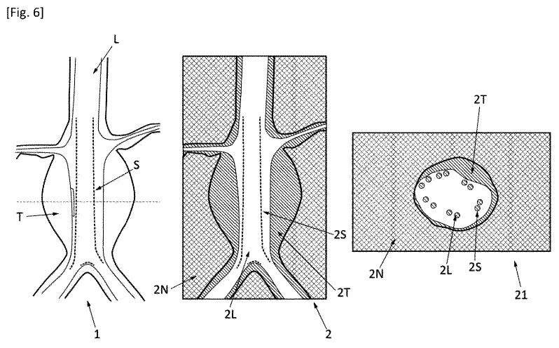

FIG. 6 shows, schematically and partially, another step of the method of FIG. 1;

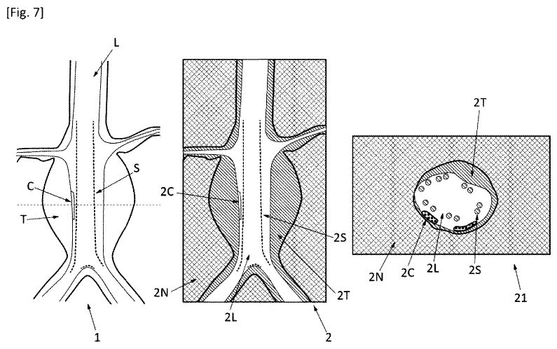

FIG. 7 shows, schematically and partially, another step of the method of FIG. 1;

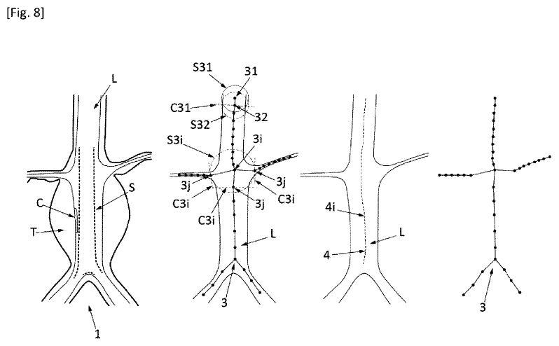

FIG. 8 shows, schematically and partially, another step of the method of FIG. 1; and

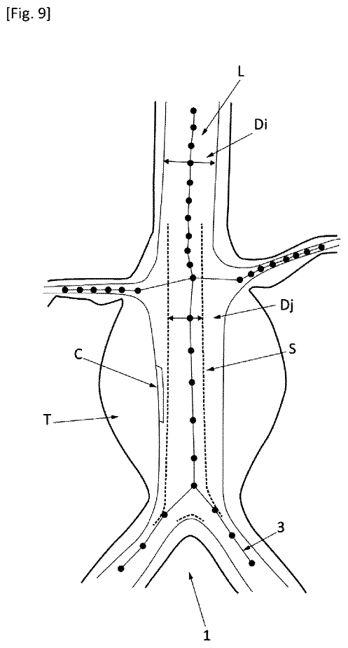

FIG. 9 shows, schematically and partially, another step of the method of FIG. 1.

In the following description, identical elements, in terms of structure or function, that appear in the various figures retain the same references unless otherwise specified. Additionally, the terms “front,” “rear,” “top” and “bottom,” “sagittal,” “axial” and “coronal” must be interpreted in the context of the orientation of the blood vessel as shown, corresponding to the orientation of the blood vessel in the human body.

FIG. 1 shows a method for aiding in the diagnosis of a cardiovascular disease of a blood vessel, in this case an aneurysm A of an abdominal aorta AA of a patient according to an exemplary embodiment of the invention.

In advance of the method, a three-dimensional representation 1 of the aorta AA of the patient was acquired by a computed tomography angiography CTA method. In this method, the patient was helically scanned by an X-ray beam so as to obtain a plurality of cross sectional images 11 of the aorta AA according to different angular incidences of the irradiating beam. Stacking these images 11, after a rotational calibration, allows digital reconstruction of a volume of voxels, forming the three-dimensional representation 1 of the aorta AA, which is provided to the method in a first step E0. Each voxel is assigned a value proportional to the absorption of the X-rays by the corresponding scanned tissue or material. This value is measured in a Hounsfield units.

For purposes of illustration, FIG. 3 to FIG. 9 show different schematic views of this three-dimensional representation 1. FIG. 3 thus shows, on the left, a cross sectional view along a coronal plane of the three-dimensional representation 1 and, on the right, an angiography 11 of the three-dimensional representation 1 located at a transverse plane X-X.

In the example described, the aorta AA has an aneurysm A, between the junction of the aorta AA and the renal arteries and the bifurcation of the aorta AA to the femoral arteries. The aorta AA thus has a lumen L wherein the blood can circulate and tunicas T (intima, media, adventitia) forming the walls of the aorta AA around the lumen L. The aneurysm A forms a thrombus around the lumen L. Moreover, it can be seen that this aneurysm A was repaired by means of a stent S placed in the lumen L, for example during angioplasty, and that a calcification C formed on the inner wall of the intima T. The described example thus corresponds to a postoperative consultation during which the practitioner is monitoring the change in the aneurysm, with the understanding that the method could be implemented during a follow-up consultation seeking to detect the aneurysm or to monitor its evolution in order to decide whether an angioplasty is wise.

During the CTA, a contrast agent or product was injected into the patient in order to improve the visibility of the aorta in the angiographies 11, and in particular to distinguish the tunicas T and the lumen L on each angiography 11. However, the boundaries between these tunicas T and the lumen L are not shown clearly on these angiographies, which further show other tissues of the patient's body outside the aorta A.

In order to be able to select only the voxels of the aorta A and to be able to clearly distinguish the boundaries between the tunicas T and the lumen L, the method comprises a step E1 of segmenting the three-dimensional representation 1. This step E1 is implemented by means of a classifier arranged to estimate whether each voxel of the three-dimensional representation 1 belongs to the aorta AA, and more precisely to the lumen L or to the tunica T, and to label this voxel as a function of this estimate.

In the example described, the classifier implements an automatic learning algorithm of the Convolutional Neural Network (CNN) type.

FIG. 2 shows an example of a CNN classifier that is particularly suitable for segmenting a three-dimensional representation of a blood vessel.

The CNN classifier of FIG. 2 comprises a contraction path CP and an expansion path EP.

The contraction path CP comprises four successive contraction blocks CB₁ to CB₄. Each contraction block comprises two convolution layers CONV, each associated with a correction layer RELU arranged to implement a rectified linear unit-type activation function, followed by a downsampling or pooling layer POOL. Each first convolution layer CONV of a block CBⱼ₊₁ thus receives the feature map from the downsampling layer POOL of the preceding block CBⱼ. The number of convolutional kernels of the convolution layers CONV of a same block CBⱼ is identical, while the number of convolutional kernels of the convolution layers of a block CBᵢ₊₁ is twice that of the block CBⱼ. It should be noted that the number of convolutional kernels of the convolution layers of the block CBi is 64. These convolutional kernels are of dimensions 3×3 and have a stride of 1. Each downsampling layer POOL comprises a mask for selecting a maximum value, of dimensions 2×2 and with a stride of 2.

The contraction path CP is connected to the expansion path EP by two convolution layers CONV, each comprising twice the convolutional kernels of the block CB₄, these kernels having dimensions 3×3 and having a stride of 1.

The expansion path EP comprises four successive expansion blocks EB₄ to EB₁. Each expansion block comprises an upsampling layer UPSAMP followed by two convolution layers CONV each associated with a correction layer RELU. Each upsampling layer UPSAMP of a block EBᵢ thus receives the feature map from the last convolution layer CONV of the preceding block EBⱼ₊₁. The number of convolutional kernels of the upsampling layers UPSAMP and convolution layer CONV of the same block EBᵢ is identical, whereas the number of convolutional kernels of the upsampling layer UPSAMP and convolution layer CONV of a block EBⱼ₊₁ is twice that of the block EBᵢ. These convolutional kernels are of dimensions 3×3 and have a stride of 1 for the convolution layers CONV and of dimensions 3×3 and have a stride of 2 for the layers UPSAMP. The output of each upsampling layer UPSAMP of a block EBᵢ is concatenated, before entering the first convolution layer of this block EBᵢ, to the feature map FM coming from the last convolution layer CONV of the contraction block CBⱼ through connection hops SC between the contraction path CP and the expansion path EP.

Finally, the CNN classifier comprises a last convolution layer CONV, receiving the feature map from the last convolution layer of the block EB₁, and comprising a convolutional kernel of size 1×1, associated with a SOFTMAX correction or normalization layer of the normalized exponential type.

Such a convolutional neural network is for example known as “U-Net.”

In the example described, this CNN U-Net classifier is a so-called “2D” network able to segment images. In a sub-step E11 of step E1, the three-dimensional representation 1 is thus scanned along three horizontal X, vertical Y and transverse Z axes to obtain a plurality of images of sagittal, axial and coronal cross sections IS, respectively, of the three-dimensional representation 1. Each of these images IS is thus segmented, in a step E12, by means of the U-Net 2D classifier, to estimate whether each pixel of the image IS belongs to the aorta AA and to label this pixel as a function of this estimate

More specifically, the CNN classifier is arranged to estimate, for each pixel of an image IS that it must segment, if:
a. this pixel is outside the aorta AA, the CNN classifier in this case allocating a first label to this pixel, for example a label of value 0,
b. this pixel belongs to the lumen L of the aorta AA, the CNN classifier in this case allocating a second label to this pixel, for example a label of value 1;
c. this pixel belongs to a T of the aorta AA, the CNN classifier in this case allocating a third label to this pixel, for example a label of value 2.

The last convolution layer CONV associated with the SOFTMAX correction layer of the CNN classifier allows the feature maps FM from the expansion path EP to be transformed into a label mask CB, allocating the label having the highest probability to each pixel of the image IS to be segmented. The label mask CB thus has dimensions identical to those of the image IS to be segmented and thus forms a two-dimensional map of this image IS.

In order to be able to correctly segment a new image IS, the CNN classifier has undergone a prior step of automatic training E01, which is said to be supervised. In this step, the CNN classifier has successively segmented a plurality of cross sectional sagittal, axial and coronal images, respectively, of a plurality of predetermined three-dimensional representations, the labels of the voxels of which are known in advance. This plurality of predetermined three-dimensional representations forms a training set TS for the CNN classifier. The CNN can thus determine, for each label that it allocates to a pixel of an image coming from this training set TS, if it has created an error and can adjust its hyperparameters automatically as a function of this error, namely the weights of the convolutional kernels of the convolution layers CONV and of the upsampling layers UPSAMP. This adjustment may for example be implemented by a gradient descent method.

In the example described, the training set TS has been artificially augmented in a preliminary step E02. In this step E02, new three-dimensional representations have been generated by modifying the spectral representations of the training set TS so as to obtain at least new three-dimensional representations that are distinct from all the three-dimensional representations of the training set TS, for example by degradation operations, resolution change operations, addition of noise, offset in one or more dimensions and/or rotation. These new three-dimensional representations were added to the training set TS. It has thus been possible, starting from a relatively limited real data set, to obtain a particularly significant training set TS so as to be able to train the CNN classifier optimally.

In a sub-step E13 of step E1, at the end of step E12, the two-dimensional maps CB obtained by the segmentation of the cross sectional sagittal, axial and coronal images IS, respectively, of the three-dimensional representation 1 were combined to form a three-dimensional map 2 of this three-dimensional representation 1.

Indeed, the pixels of the cross sectional images IS can be positioned in space, the coordinates of the cross sectional images IS being known due to the scanning. Therefore, each label associated with a pixel can be repositioned in space, so as to recombine all of the two-dimensional maps CB to form voxels of labels, this set of voxels of labels then forming the three-dimensional map 2.

In the example described, when two labels, associated with pixels of cross sectional images IS obtained along two distinct axes and which correspond to the same voxel, have distinct values, the label with the highest value among these two labels is assigned to this voxel.

Thus, in FIG. 4, on the left, the cross sectional view along a coronal plane of the three-dimensional representation 1 is shown, as shown in FIG. 3; at the center, a cross sectional view along the same coronal plane of the three-dimensional map 2 is shown; and, on the right, a cross section 21 of the three-dimensional map 2 located at a transverse plane X-X is shown.

It is thus observed that the three-dimensional representation 1 has indeed been segmented, at the three-dimensional map 2, into three volumes of points 2N, 2L and 2T, corresponding respectively to the labels of values 0, 1 and 2. However, the segmentation performed by the CNN is a statistical method, which can introduce errors.

In order to detect, and if necessary, to correct these errors, the method comprises a step E2 of confirming and correcting the labels assigned by the CNN classifier to the voxels of the three-dimensional representation 1.

This step E2 consists, in the example described, on the one hand, in carrying out morphological operations of the erosion and expansion type on the three-dimensional map 2, and, on the other hand, in propagating the label of the voxels of a zone of the three-dimensional map 2 to the voxels of another zone whose label is different but whose texture is substantially identical.

In the example described, the method thus comprises a sub-step E21 for determining averages of intensity gradients and/or average intensities of first zones Z1 of voxels of the three-dimensional representation 1 to which the first label of value 0 was allocated and second zones Z2 of voxels of the three-dimensional representation 1 to which the second label of value 1 has been allocated, these zones Z1 and Z2 being located on either side of a boundary separating the volume of points 2T and the volume of points 2N in the three-dimensional map 2. In the case where a first zone Z1 and a neighboring second zone Z2 have the same average intensity and/or a same average intensity gradient from one zone to another, the second label of value 1 will be allocated to the voxels of the first zone Z1. In this example, two average gradients or intensities are considered identical if the absolute value of their difference is less than a threshold proportional to the standard deviation of these gradients and of these intensities.

The method also comprises, following step E21, a sub-step E22 of determining average intensity gradients and/or average intensities of second zones Z2 of voxels of the three-dimensional representation 1 to which the second label of value 1 has been allocated and third zones Z3 of voxels of the three-dimensional representation 1 to which the third label of value 2 has been allocated, these zones Z2 and Z3 being located on either side of a boundary separating the volume of points 2T and the volume of points 2L in the three-dimensional map 2. In the case where a second zone Z2 and a neighboring third zone Z3 have the same average intensity and/or a same average intensity gradient from one zone to another, the third label of value 2 will be allocated to the voxels of the second zone Z2.

Thus, in FIG. 5, on the left, the cross sectional view along a coronal plane of the three-dimensional representation 1 is shown; at the center, the cross sectional view along the same coronal plane of the three-dimensional map 2 at the end of step E2; and, on the right, a cross section 21 of the three-dimensional map 2 located at a transverse plane X-X.

It is thus observed that a second zone Z2, previously labeled by the CNN classifier as being part of the tunica, has been corrected, its label now being the third label of value 2, thus indicating that the voxels of this zone Z2 in the three-dimensional representation 1 actually form part of the lumen L. Although the volumes of points 2N, 2L and 2T are now reliable, certain voxels of the three-dimensional representation 1 require a particular label in order to identify the stent S and the calcification C.

For these purposes, the method comprises a step E3 of comparing the value of each voxel of a plurality of voxels of the three-dimensional representation 1, the labels of which allocated to the three-dimensional map 2 are those of the aorta, namely the labels of values 1 and 2, with a predetermined threshold value, a label different from those of the blood vessel being allocated to each voxel whose value exceeds said predetermined threshold value.

More specifically, in the example described, the comparison step E3 comprises a first sub-step E31 of comparing the value of each voxel whose allocated label on the three-dimensional map 2 is the third label of value 2 and which is located at a boundary separating the volume of points 2T and the volume of points 2L in the three-dimensional map 2, with a first predetermined threshold value. A label, for example of value 3, associated with a stent, is allocated to each voxel whose value exceeds said first predetermined threshold value.

The comparison step E3 also comprises a second sub-step E32 of comparing the value of each voxel whose allocated label on the three-dimensional map 2 is the third label of value 2 and which is located at a boundary separating the volume of points 2T and the volume of points 2L in the three-dimensional map 2, with a second predetermined threshold value that is less than the first threshold value. A label, for example of value 4, associated with calcification, is allocated to each voxel whose value exceeds said second predetermined threshold value.

Thus, in FIG. 6, on the left, the cross sectional view along a coronal plane of the three-dimensional representation 1 is shown; at the center, the cross sectional view along the same coronal plane of the three-dimensional map 2 at the end of step E31; and, on the right, a cross section 21 of the three-dimensional map 2 located at a transverse plane X-X.

The appearance, in the three-dimensional map, of voxels 2S is observed in the volume of points 2L, in the vicinity of the boundary of this volume of points 2L with the volume of points 2T, which have a label distinct from that of the lumen L. These are thus voxels 2S corresponding to the stent S.

FIG. 7 also shows, on the left, the cross sectional view along a coronal plane of the three-dimensional representation 1; at the center, the cross sectional view along the same coronal plane of the three-dimensional map 2 at the end of step E32; and, on the right, a cross section 21 of the three-dimensional map 2 located at a transverse plane X-X.

The appearance, in the three-dimensional map, of voxels 2C is observed in the volume of points 2L, in the vicinity of the boundary of this volume of points 2L with the volume of points 2T, which have a label distinct from that of the lumen L. These are thus voxels 2C corresponding to calcification C.

At the end of step 3, a three-dimensional map 2 of the aorta AA is thus available reliably identifying all voxels of the three-dimensional representation 1 belonging to the same element of the aorta.

The method comprises a step E4 of determining the evolution of a geometric indicator of the aorta along this blood vessel, by means of the voxels of the three-dimensional representation 1, the labels of which allocated to the three-dimensional map 2 are those of the blood vessel.

In the example described, this step E4 is a step of determining the evolution of the actual diameter of the lumen L of the aorta, that is, the diameter effectively allowing the circulation of blood.

In a sub-step E41, a graph 3 is estimated passing through the entire aorta AA, and each point 3i of which is the barycenter of the voxels located in a cross section of the three-dimensional representation 1 locally orthogonal to the graph and whose labels are those of the lumen L.

For these purposes, the first point of the graph 31 corresponds to the barycenter of the voxels located in the highest coronal cross section of the three-dimensional representation 1 and whose labels are those of the lumen L of the aorta AA.

A sphere S31 is positioned on this first point 31, the radius of this sphere S31 being such that the sphere S31 encompasses all the voxels located in the coronal cross section of the three-dimensional representation 1 passing through the point 31 and whose labels are those of the lumen L.

This sphere S31 intersects the lumen L at an intersection C31, the barycenter of which is then determined, which forms the second point 32 of the graph 3.

Each point along 3j of the graph can thus be determined by estimating the barycenter of the intersection between the lumen L and a sphere S3i, centered on the preceding point 3i of the graph 3 and with a radius greater than or equal to the smallest radius encompassing the voxels situated in the cross section C3i of the lumen L passing through the preceding point 3i of the graph 3 and whose labels are those of the lumen L, until the entire three-dimensional representation 1 has been traveled.

It can be seen in particular that this step E41 allows, when the aorta AA has a branch, identification of an intersection C3i between the sphere S3i and each branch of the aorta. It is then possible to duplicate the algorithm to travel each of these branches, from the barycenter 3j of each of these intersections.

The set of points 3i thus forms a graph 3 where each point is connected to at least one other point of the graph, so that it is possible to travel the aorta AA from end to end by means of the graph 3.

In a sub-step E42, the gradient of the signed distance function to the walls of the lumen L of the aorta AA, that is, the boundary separating the volume of points 2L from the volume of points 2T, is also estimated determined.

The barycenters of the discontinuity points of this gradient, for example determined in each cross section orthogonal to the aorta AA according to the graph 3, thus form a point cloud 4, each point 4i of the point cloud 4 being the point locally furthest from this boundary.

In a step E43, the point 4i of the point cloud 4 closest to this point 3i is determined for each point 3i of the graph 3, by a least squares method. The spatial coordinates of the point 3i of the graph 3 are then replaced by those of the point 4i. It should be noted that this replacement is dependent on the fact that the spatial coordinates of the point 4i substantially satisfy an equation representing the branch of the graph 3 on which the point 3i is positioned.

The graph 3, at the end of step E43, thus makes it possible to travel the entire aorta AA while representing the points of the lumen L furthest from the walls of this lumen L.

FIG. 8 shows, successively from left to right:
a. the cross sectional view along a coronal plane of the three-dimensional representation 1;
b. a cross sectional view of the lumen L along the same coronal plane of the three-dimensional map 2, including the graph 3 determined at the end of step E41;
c. a cross sectional view of the lumen L along the same coronal plane of the three-dimensional map 2, including the point cloud 4 determined at the end of step E42;
d. a cross sectional view of the graph 3 according to the same coronal plane of the three-dimensional map 2, determined at the end of step E43.

Each of the points 3i of the graph 3 thus allows determination, in a step E5, of the local diameter Di of the lumen L, in a cross section of this lumen L locally orthogonal to the graph 3 passing through this point 3i, this diameter Di being the diameter of the lumen L between its walls if this cross section is free of voxels whose labels are those of the stent S or the calcification C, or otherwise, a diameter of the lumen L taking into account these voxels whose labels are those of the stent S or the calcification C.

Thus, in FIG. 9 the cross sectional view along a coronal plane of the three-dimensional representation 1 is shown, as shown in FIG. 1, to which the graph 3 and two local diameters Di and Dj were added, determined at the end of step E5.

The foregoing description clearly explains how the invention makes it possible to achieve the objectives that it has set, namely, to be able to estimate an actual geometric indicator of a blood vessel in a simple, reliable, rapid, reproducible and non-practitioner-dependent manner, by proposing a method wherein a three-dimensional representation of the blood vessel is automatically segmented, by means of the classifier, so as to be able to exclusively select the voxels of this representation which actually correspond to the blood vessel, and then wherein the three-dimensional representation is processed to identify, by thresholding, the voxels classified by error by the classifier as belonging to the vessel whereas they correspond to a stent arranged in the vessel or to calcification of the vessel.

In any case, the invention is not limited to the embodiments specifically described in this document, and extends in particular to any equivalent means and to any technically operative combination of these means. In particular, it is possible to envisage using the method on other types of three-dimensional representation of a blood vessel, such as, for example, those resulting from other medical imaging techniques, such as magnetic resonance imaging, or as those derived from computed tomography angiography without contrast agent.

Other types of classifier can also be envisaged for segmenting the three-dimensional representation, such as for example a classifier of the “U-Net 3D” type able to directly segment the three-dimensional representation in order to obtain said three-dimensional map, using three-dimensional convolutional kernels. It will also be possible to envisage using other types of convolutional neural networks, or even other types of classifier implementing an automatic learning algorithm.

It is also possible to envisage determining, alternatively or cumulatively, the evolution of other geometric indicators of the blood vessel such as its diameter, and in particular its volume.

1. A method for aiding in the diagnosis of a cardiovascular disease of a blood vessel, comprising the following steps:
a. providing a three-dimensional representation of a blood vessel of a patient, obtained by a medical imaging device;
b. segmenting, by means of a classifier, said three-dimensional representation to obtain a segmented three-dimensional map of said three-dimensional representation, the classifier being arranged to estimate whether each voxel of the three-dimensional representation belongs to said blood vessel and to label this voxel as a function of this estimate, said segmented three-dimensional map being formed by the set of labels assigned by the classifier to the voxels of the three-dimensional representation;
c. comparing the value of each voxel of a plurality of voxels of the three-dimensional representation, the allocated labels on the three-dimensional map of the voxels being those of the blood vessel, with a predetermined threshold value, a label different from those of the blood vessel being allocated to each voxel with a value that exceeds said predetermined threshold value;
d. determining the change in a geometric indicator of the blood vessel along this blood vessel by means of the voxels of the three-dimensional representation, the allocated labels on the three-dimensional map of the aforementioned voxels being those of the blood vessel.

2. The method according to claim 1, wherein the classifier is arranged to estimate, for each voxel of the three-dimensional representation, whether:
a. this voxel is outside the blood vessel, the classifier in this case allocating a first label to this voxel,
b. this voxel belongs to the lumen of the blood vessel, the classifier in this case allocating a second label to this voxel,
c. this voxel belongs to a tunica of the blood vessel, the classifier in this case allocating a third label to this voxel.

3. The method according to claim 2, wherein the segmentation step is implemented by a classifier implementing a machine learning algorithm.

4. The method according to claim 3, wherein the classifier is a convolutional neural network, comprising a contraction path and an expansion path, wherein the contraction path comprises a plurality of convolution layers each associated with a correction layer arranged to implement an activation function and downsampling layers, each downsampling layer being followed by at least one convolution layer, wherein the expansion path comprises a plurality of convolution layers and upsampling layers, each upsampling layer being followed by a convolution layer.

5. The method according to claim 4, wherein the output of each upsampling layer is concatenated, before entering the next convolution layer, to the feature map arising from a corresponding convolution layer of the contraction path through a connection hop between the contraction path and the expansion path.

6. The method according to claim 3, wherein the segmentation step comprises the segmentation by means of the classifier of three axial, sagittal and coronal cross sections of said three-dimensional representation to obtain three segmented two-dimensional maps and a step of combining the two-dimensional maps to obtain said three-dimensional map.

7. The method according to claim 1, wherein it comprises, at the end of the segmentation step and prior to the comparison step, a step of confirming and correcting the labels allocated by the classifier to the voxels of the three-dimensional representation.

8. The method according to claim 1, wherein the comparison step comprises:
a. a first sub-step of comparing the value of each voxel of a plurality of voxels of the three-dimensional representation, the allocated labels on the three-dimensional map of the voxels being those of the blood vessel, with a first predetermined threshold value, a first label associated with a stent being allocated to each voxel with a value that exceeds said first predetermined threshold value;
b. a second sub-step of comparing the value of each voxel of a plurality of voxels of the three-dimensional representation, the allocated labels on the three-dimensional map of the voxels being those of the blood vessel, with a second predetermined threshold value that is less than the first threshold value, a second label associated with calcification being allocated to each voxel with a value that exceeds said second predetermined threshold value.

9. The method according to claim 1, wherein the comparison step is implemented for a plurality of voxels whose allocated labels on the three-dimensional map are those of the lumen of the blood vessel and are located at a boundary of the three-dimensional map between the labels of the lumen and the labels of the tunicas of the blood vessel.

10. The method according to claim 1, wherein the step of determining the evolution of a geometric indicator of the blood vessel is a step of determining the evolution of the diameter of the blood vessel and comprises a step of estimating a graph traveling the entire blood vessel and each point of which is the barycenter of the voxels located in a cross section of the three-dimensional representation locally orthogonal to the graph and the labels of which are those of the blood vessel; wherein a local diameter of the blood vessel is determined as a function of each of the points of the graph.

11. The method according to claim 10, wherein the determining step further comprises a step of estimating a point cloud, each point of the point cloud being the point locally furthest from a boundary of the voxels of the three-dimensional representation of the blood vessel and the labels of which are those of the blood vessel, and a step of correcting the points of the graph using the point cloud.
